(19) United States
(12) Patent Application Publication
(10) Pub. No.: US 2013/0345440 A1
(43) Pub. Date: Dec. 26, 2013

(54) CARBOXYLATION OF POLY-/OLIGOTHIOPHENES

(71) Applicants: Christian Severins, Leverkusen (DE); Asier Eleta-Lopez, Lekunberri (ES); Sigurd Buchholz, Koln (DE); Kilian Tellmann, Frankfurt am Main (DE); Björn Henninger, Hamburg (DE); Leslaw Mleczko, Dormagen (DE)

(72) Inventors: Christian Severins, Leverkusen (DE); Asier Eleta-Lopez, Lekunberri (ES); Sigurd Buchholz, Koln (DE); Kilian Tellmann, Frankfurt am Main (DE); Björn Henninger, Hamburg (DE); Leslaw Mleczko, Dormagen (DE)

(73) Assignee: Bayer Intellectual Property GmbH, Monheim (DE)

(21) Appl. No.: 13/993,366

(22) PCT Filed: Dec. 8, 2011
(86) PCT No.: PCT/EP11/72172

§ 371 Date: Sep. 13, 2013

(30) Foreign Application Priority Data
Dec. 13, 2010 (DE) 10 2010 062 961.8

(51) Int. Cl. C07D 333/38

(52) U.S. Cl. CPC C07D 333/38
USPC 549/59

(57) ABSTRACT

The present invention relates to a process for carboxylation of poly/oligothiophenes using CO₂.

The present invention relates to a process for carboxylation of poly/oligothiophenes using CO₂.

In the last 15 years, the field of molecular electronics has developed rapidly with the discovery of organic conductive and semiconductive compounds. Within this period, a multitude of compounds having semiconductive or electrooptical properties has been found. It is generally accepted that molecular electronics will not displace conventional semiconductor units based on silicon; instead, it is assumed that molecular electronic components will open up new fields of use in which suitability for coating large areas, structural flexibility, processability at low temperatures and low costs are important. Semiconductive organic compounds are currently being developed for fields of use such as organic field-effect transistors (OFETs), organic luminescent diodes (OLEDs), sensors and photovoltaic elements.

The known conductive or semiconductive organic compounds generally have consecutive conjugated units and are divided according to molecular weight and structure into conjugated polymers and conjugated oligomers. Oligomers are generally distinguished from polymers in that oligomers usually have a narrow molecular weight distribution and a molecular weight up to about 10 000 g/mol (Da), whereas polymers generally have a correspondingly higher molecular weight and a broader molecular weight distribution. However, a more sensible distinction is on the basis of the number of repeat units, since a monomer unit can quite possibly reach a molecular weight of 300 to 500 g/mol, for example in the case of (3,3″″-dihexyl)quaterthiophene. In the case of a distinction according to the number of repeat units, reference is still made to oligomers within the range from 2 to about 20. However, there is a fluid transition between oligomers and polymers.

The most important semiconductive organic compounds include the poly/oligothiophenes, the monomer unit of which is, for example, 3-hexylthiophene.

Processes for synthesis of oligo/polythiophenes are described, for example, in EP1026138A, EP2121798A, EP2121799A, WO09/015810A and WO09/021639A.

Organic/inorganic hybrid solar cells based on conductive organic polymers as electron donors, for example poly(3-hexylthiophene) (P3HT), and inorganic semiconductor nanoparticles, for example CdSe nanoparticles, are known from the prior art (see, for example, N. C. Greenham, X. Peng, and A. P. Alivisatos, Physical Review B 54, 17628 (1996); X. Peng, L. Manna, W. Yang, J. Wickham, E. Scher, A. Kadavanich, A. P. Alivisatos, Nature 404, 59 (2000)).

The performance of a solar cell depends upon factors including the solubility and surface characteristics of the nanoparticles—properties which can considerably influence electron transfer between semiconductive polymer and nanoparticles and between the individual nanoparticles. Often, in the case of production of nanoparticles, ligands having long alkyl radicals are used, these being intended to prevent aggregation of nanoparticles. In the solar cell, however, these ligands with alkyl radicals have an adverse effect, since they can lead to electrical passivation of the nanoparticles.

In order to improve charge transfer in hybrid solar cells, it is customary to exchange the ligands around the nanoparticles after the synthesis thereof. The treatment of nanoparticles with pyridine is an effective method frequently described in the literature for increasing the efficiency of a solar cell (see, for example, Olson et al., Solar Energy Materials & Solar Cells 93, 519 (2009)).

D. J. Milliron et al. describe electroactive surfactants, for example pentathiophenephosphonic acid (T5-PA), which are used in a ligand exchange for complexation of CdSe nanoparticles, in order to improve charge transfer between semiconductive polymer and nanoparticles (Adv. Mater. 2003, 15, No. 1, Pages 58-61).

A publication by T. Antoun et al. (Eur. J. Inorg. Chem. 2007, Pages 1275-1284) describes CdS nanoparticles functionalized by electroactive carboxylated oligothiophenes. The binding of the oligothiophenes via a carboxyl group to the CdS nanoparticle improved the electronic interaction between oligothiophene and nanoparticles. Carboxylated oligothiophenes which are used as surfactants in hybrid solar cells are therefore of interest for optoelectronic and photovoltaic applications. The same applies to carboxylated polythiophenes, for which, through the carboxylation, an improved electronic interaction with semiconductive nanoparticles can likewise be expected.

The carboxylation of heteroaromatic compounds is typically effected in a two-stage process consisting of an acylation of the heteroaromatic with subsequent oxidation to give the corresponding carboxylate compounds. Typically, the corresponding Friedel-Crafts acylations are performed in the presence of stoichiometric amounts of Lewis acids in anhydrous solvents (see, for example, DE102007032451A1, EP178184A1).

The conversion of such reactions from laboratory to an industrial scale always constitutes a considerable problem, since the solvents are environmentally harmful in different ways. The product isolation also gives rise to relatively large amounts of wastewater with a high salt content, which have to be worked up. The oxidation of the aryl ketone is typically performed with organic peroxides or inorganic oxidizing agents (Dodd et al. Synthesis 1993, 295-297; U.S. Pat. No. 5,739,352). The conversion of such reactions to the industrial scale likewise constitutes a considerable problem, since the oxidizing agents are environmentally harmful in different ways and the reactions are strongly exothermic.

An efficient preparation method for aromatic and heteroaromatic carboxylate compounds is what is called direct carboxylation with CO₂. CO₂ is additionally a nontoxic and readily available, inexpensive C₁ source. Nevertheless, there are only a few literature examples for the direct carboxylation of aromatics and heteroaromatics with CO₂.

U.S. Pat. No. 2,948,737 describes such a direct carboxylation of heteroaromatics. It is disclosed therein that the direct carboxylation with gaseous CO₂ succeeds with moderate yields (8%) at temperatures of >300° C. in the presence of acid-binding reagents at a reaction pressure of 1570 bar in an autoclave.

U.S. Pat. No. 3,138,626 states that direct carboxylation with gaseous CO₂ can be performed with moderate yields (22%) from temperatures of 100° C. in the presence of AlCl₃ at a reaction pressure of 200 bar in an autoclave.

Owing to the high reaction temperatures, the conversion of such reactions to the industrial scale constitutes a considerable problem, since many carboxylic acids of aromatics and heteroaromatics have much lower decomposition temperatures.

Ohishi et al. (Angew. Chem. Int. Ed. 2008, 47, 5792-5795) describe experiments in which aromatic and heteroaromatic carboxylic acids were prepared in organic solvents using mixtures consisting of boronic esters, a homogeneous copper-carbene catalyst and CO₂ at distinctly lower temperatures (70° C.).

Oshima et al. (Org. Lett., 2008, 10, 2681-2683) disclose experiments in which aromatic carboxylic acids were prepared at room temperature using mixtures consisting of organic zinc compounds, a homogeneous nickel-phosphorus catalyst and gaseous CO₂.

A problem with the conversion of these reactions to an industrial process is the use of costly homogeneous catalysts which are not recyclable. The product isolation also gives rise to relatively large amounts of wastewater with a high salt content, which have to be worked up.

In summary, it can be stated that there is a need for an inexpensive process performable in a simple manner for carboxylation of poly- and oligothiophenes, which can also be performed on the industrial scale.

Proceeding from the known prior art, the technical problem addressed is thus that of providing a process for carboxylation of poly- and oligothiophenes, which is comparatively easy to perform and inexpensive and leads to higher yields. The process sought shall additionally have minimum potential to damage the environment and reliable temperature control. The formation of large amounts of salt-containing wastewater shall be avoided. The process shall especially be usable for preparation of 3,3′″-dihexyl-2,2′:5′,2″:5″,2′″-quaterthiophene-5-carboxylic acid.

According to the invention, this problem is solved by a process according to claim 1. Preferred embodiments can be found in the dependent claims.

The process according to the invention for carboxylation of poly- and oligothiophenes comprises at least the following steps:
a) providing a first liquid component comprising a poly- and/or oligothiophene,
b) providing a second liquid component comprising an organic and/or inorganic base,
c) mixing the first and second liquid components,
d) mixing the mixture from step c) with CO₂ to react the aromatic or heteroaromatic compounds with CO₂.

In step a) of the process according to the invention, a first liquid component at least comprising a poly- and/or olgiothiophene is provided.

Preference is given to using an oligothiophene having a chain length of ≧2 to ≦20 monomer units, preferably of ≧3 to ≦12, more preferably of ≧4 to ≦10 and most preferably of ≧5 to ≦8 monomer units.

In a particularly preferred embodiment of the present invention, 3,3′″-dihexylquaterthiophene is used as the reactant.

The reactant is provided in liquid form. The reactant (thiophene) may already be present in liquid form. In this case, the component referred to as the first liquid component in step a) may be the liquid reactant. It is likewise conceivable to first dissolve the reactant in a solvent and to provide this solution as the first liquid component.

Carboxylation is understood to mean the introduction of a carboxyl group into an organic compound. Carboxylation is a reaction for preparation of carboxylic acids.

In step b) of the process according to the invention, a second liquid component at least comprising an organic and/or inorganic base is provided. The second liquid component may be the base itself; it is likewise conceivable that the second liquid component is a solution in which an organic and/or inorganic base is present.

The base used is preferably n-butyllithium, t-butyllithium, methyllithium, phenyllithium, lithium diisopropylamide (LDA) and/or hexyllithium.

In step c) of the process according to the invention, the first and second liquid components are mixed. The liquid components are preferably combined at a temperature in the range from −100° C. to 40° C. and at a pressure of 1 to 60 bar.

The aim of step c) is to obtain a very homogeneous mixture of the two liquid components.

In step d) of the process according to the invention, the mixture obtained from step c) is mixed with CO₂. CO₂ can be added in the gaseous, liquid, solid or supercritical state or in solution to the mixture of the base and the aromatic and/or heteroaromatic. Preference is given to adding CO₂ in the gaseous or liquid state.

The mixing in step d) is effected preferably at a temperature in the range from −100° C. to 60° C. and at a pressure of 1 to 60 bar.

The addition of CO₂ initiates the carboxylation of the thiophene compound. The reaction between the poly- and/or oligothiophene with CO₂ is performed up to the desired or achievable conversion.

Preference is given to working up the reaction mixture after the conversion of the reactants in order to isolate and optionally to purify the desired carboxylated product. The process according to the invention therefore preferably comprises a further step e) after step d):

e) collecting the mixture from step d) and isolating the carboxylated product.

For isolation of the carboxylated thiophene, the reaction mixture is preferably first provided with acid in order to bind amounts of base still present. The carboxylated product can be isolated, for example, by extraction and/or distillation and/or chromatography.

The process according to the invention can be executed continuously or batchwise. It is likewise conceivable to execute some steps of the process according to the invention continuously and the other steps discontinuously. Preference is given to performing at least steps c) and d) continuously.

Continuous steps in the context of the invention are those in which the feed of compounds (reactants) into a reactor and the discharge of compounds (products) from the reactor take place simultaneously but spatially separately, whereas, in discontinuous steps, the sequence of feeding in compounds (reactants), optional chemical conversion and discharge of compounds (products) proceeds successively in time. The continuous procedure is economically advantageous since periods for which reactors are idle as a result of filling and emptying processes and long reaction times as a result of safety requirements, reactor-specific heat exchange performances and heating and cooling processes as occur in batchwise processes (discontinuous processes) are avoided.

The preferably continuous mixing of compounds in step c) and/or in step d) is preferably performed by means of a static mixer.

While the homogenization of a mixture is achieved by means of moving equipment, for example stirrers, in the case of dynamic mixers, the flow energy of the fluid is exploited in the case of static mixers: a conveying unit (for example a pump) forces the liquid, for example, through a tube provided with static mixer internals, the liquid which follows the main flow axis being divided into component streams which are vortexed and mixed with one another according to the type of internals.

An overview of different types of static mixers as used in conventional chemical engineering is given, for example, by the article “Statische Mischer und ihre Anwendungen” [Static mixers and their uses], M. H. Pahl and E. Muschelknautz, Chem.-Ing.-Techn. 52 (1980) No. 4, p. 285-291.

An example given here for usable static mixers is that of SMX mixers (cf. patent U.S. Pat. No. 4,062,524). They consist of two or more mutually perpendicular grids of parallel strips which are bonded to one another at their crossing points and are set at an angle to the main flow direction of the mixture, in order to divide the liquid into component streams and to mix it. A single mixing element is unsuitable as a mixer since mixing occurs only along a preferential direction transverse to the main flow direction. Therefore, several mixing elements rotated by 90° with respect to one another have to be arranged in succession.

For the process according to the invention, or for steps of the process according to the invention, the use of micro process technology is advantageous.

Modular micro process technology or micro reaction technology makes it possible to combine various micro process modules according to a modular principle to give a complete microscale production plant.

Modular micro reaction systems are supplied commercially, for example by Ehrfeld Mikrotechnik BTS GmbH. The commercially available modules include mixers, reactors, heat exchangers, sensors and actuators, and many more.

Preference is given to mixing in step c) and/or step d) by means of one or more “micro mixers”.

The term “micro mixer” used represents microstructured, preferably continuous reactors known by the name of micro reactor, mini reactor, micro heat exchanger, mini mixer or micro mixer. Examples are micro reactors, micro heat exchangers, T and Y mixers, and micro mixers from a wide variety of companies (for example Ehrfeld Mikrotechnik BTS GmbH, Institut für Mikrotechnik Mainz GmbH, Siemens AG, CPC-Cellular Process Chemistry Systems GmbH, and others), as are common knowledge to those skilled in the art, a “micro mixer” in the context of the present invention typically having characteristic/determining internal dimensions of up to 1 mm and containing static mixing internals. An example of a static micro mixer is the rhombic mixer described in DE20219871U1.

As a result of the reduction in the characteristic dimensions, as well as heat transfer operations, mixing operations in micro mixers also proceed much more rapidly than in conventional mixers. Thus, the processing speeds in micro mixers are in some cases several powers of ten higher than in conventional apparatuses, and the mixing distances are reduced to a few millimeters.

Preference is given to converting a poly- and/or oligothiophene in step d) of the process according to the invention by conducting the reaction mixture through a delay zone. The delay zone preferably has one or more static mixers.

The metering rates of all components and the flow rate of the reaction mixture through the delay zone depend primarily on the desired residence times or conversions to be achieved. The higher the maximum reaction temperature, the shorter the residence time should be. In general, the reactants in the reaction zone have residence times between 20 seconds (20 sec) and 400 minutes (400 min), preferably between 1 min and 400 min, most preferably between 1 min and 20 min.

The residence time can be controlled, for example, by the volume flow rates and the volume of the reaction zone. The reaction profile is advantageously monitored by means of various measurement devices. Suitable for this purpose are especially devices for measurement of temperature, of viscosity, of thermal conductivity and/or of refractive index in the flowing media and/or devices for measurement of infrared and/or near infrared spectra.

It is conceivable to supply CO₂ to the reaction mixture along part of the delay zone or along the entire delay zone.

The process according to the invention can preferably be executed in temperature-controllable flow reactors. In a preferred embodiment, the reaction system for performance of the process according to the invention comprises at least two zones with independently controllable temperatures. In the first zone, the liquid components are mixed, comprising an aromatic and/or heteroaromatic compound and an inorganic and/or organic base (step c)). In the second zone, the reaction zone, CO₂ is added and the aromatic and/or heteroaromatic compound is converted (step d)). At the end of the reaction zone, the product is preferably captured and collected in order to isolate the desired product in a downstream step (step e)).

The invention is illustrated in detail below by examples, but without any restriction thereto.

### Example 1

### Synthesis of 3,3′″-dihexylquaterthiophene-1-carboxylic Acid from 3,3′″-dihexylquaterthiophene

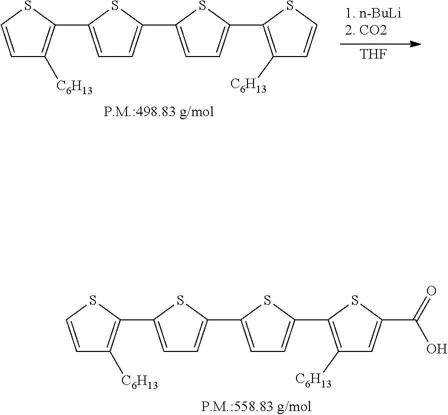

A solution of 10 parts by mass of 3,3′″-dihexylquaterthiophene and 90 parts by mass of THF was introduced into reservoir 1. A solution of 23 parts by mass of n-butyllithium and 77 parts by mass of hexane was introduced into reservoir 2. The two reservoirs were connected by a preliminary temperature control zone (0° C.) to a static mixer (volume 0.3 ml), the outlet duct of which was connected to a delay element which had a volume of 5.3 cm³ and a ratio of surface to volume of 26.3 cm²/cm⁻³ (0° C.) and led into an inlet of a further static mixer (volume 0.3 ml). Connected at the second inlet of the static mixer via a pressure-reducing valve (1.3 bar) was a CO₂ gas bottle, and the outlet duct of the static mixer was connected to a delay element having a volume of 1300 cm³ and a ratio of surface to volume of 40 cm²/cm⁻³ (0° C.). The solution from reservoir 1 was pumped continuously through the reactor at a volume flow rate of 427 ml/h and the solution from reservoir 2 at a volume flow rate of 34 ml/h. The total residence time was 33 min. The reaction was monitored regularly by HPLC. The relative yield of 3,3′″-dihexylquaterthiophene-1-carboxylic acid was >80%. The product stream was quenched at 0° C. to 5.7 M HCl solution. After phase separation and washing of the aqueous phase with n-hexane, the combined organic phases were concentrated to dryness.

1-10. (canceled)

11. A process for carboxylating a poly- and/or oligothiophene, comprising
a) providing a first liquid component comprising a poly- and/or oligothiophene,
b) providing a second liquid component comprising an organic and/or inorganic base,
c) mixing the first and second liquid components,
d) mixing the mixture from step c) with CO₂ to form a reaction mixture and reacting the aromatic or heteroaromatic compound with CO₂ to form a product mixture comprising a carboxylated product.

12. The process of claim 11, comprising a further step e) after step d),
e) collecting the product mixture from step d) and isolating the carboxylated product.

13. The process of claim 11, wherein step c) and/or step d) is/are performed continuously.

14. The process of claim 11, wherein the mixing in step c) and/or in step d) is performed by means of a static mixer.

15. The process of claim 11, wherein the reaction of CO₂ with a poly- and/or oligothiophene is performed in a micro reaction system.

16. The process of claim 11, wherein the poly- and/or oligothiophene is 3,3′″-dihexylquaterthiophene.

17. The process of claim 11, wherein the inorganic and/or organic base comprises at least one compound selected from the group consisting of n-butyllithium, t-butyllithium, methyllithium, phenyllithium, lithium diisopropylamide (LDA), and hexyllithium.

18. The process of claim 11 wherein CO₂ is added in the gaseous or liquid state.

19. The process of claim 11, wherein the reaction mixture in step d) is conducted through a delay zone, the delay zone having one or more static mixers.

20. The process of claim 19, wherein the reaction mixture in step d) resides for a residence time in the range from 20 seconds to 400 minutes in the delay zone.
